(19) United States
(12) Patent Application Publication
(10) Pub. No.: US 2025/0127985 A1
(43) Pub. Date: Apr. 24, 2025

(54) IMPLANTABLE AND REFILLABLE DRUG DELIVERY RESERVOIR SYSTEM WITH POROUS METAL FRIT FOR SUSTAINED INTRACEREBROVENTRICULAR DELIVERY AND METHODS OF USE

(71) Applicant: Genentech, Inc., South San Francisco, CA (US)

(72) Inventors: Mayumi Naito Bowen, South San Francisco, CA (US); Joshua David Horvath, South San Francisco, CA (US); Devon Michael Thompson, South San Francisco, CA (US); Darren Gilbert Doud, South San Francisco, CA (US)

(21) Appl. No.: 19/000,052

(22) Filed: Dec. 23, 2024

Related U.S. Application Data
(63) Continuation of application No. PCT/US2023/069276, filed on Jul. 1, 2022.
(60) Provisional application No. 63/357,819, filed on Jul. 1, 2022.

(51) Int. Cl. A61M 5/168; A61K 31/7088

(52) U.S. Cl. CPC A61M 5/16804; A61K 31/7088; A61M 2202/0464; A61M 2205/04

(57) ABSTRACT

A device for delivery of a drug into cerebrospinal fluid of a brain including a hollow container having a reservoir volume and an outlet, the container having an upper surface and a lower surface; a porous structure having a sintered rigid material that passively regulates diffusion of a drug from the container at a controlled release rate; and a catheter coupled to a lower end of the container, the catheter having a lumen, a proximal end region, and a distal end region, the lumen configured to communicate with the reservoir volume of the container through the porous structure. Related systems and methods are provided.

### CROSS-REFERENCE TO RELATED APPLICATIONS

This application is a continuation of PCT/US2023/069276 filed Jun. 28, 2023, which claims the benefit of U.S. Provisional Patent Application No. 63/357,819, filed Jul. 1, 2022, the content of which is hereby incorporated by reference herein in its entirety.

### FIELD

The present technology relates generally to implantable and refillable drug delivery reservoir systems and more particularly, to refillable implantable drug delivery systems having a porous metal frit for sustained delivery into the central nervous system.

### BACKGROUND

Delivery of therapeutic agents into the central nervous system (CNS) via routine systemic administrations such as oral, intravenous, subcutaneous administrations presents a challenge due to the blood-brain barrier and blood-cerebrospinal fluid barriers. CNS-directed deliveries of therapeutic agents via cerebrospinal fluid (CSF) with intracerebroventricular (ICV), intrathecal cisterna magna (IT-CM), and intrathecal-lumbar (IT-L) administrations are considered a more effective alternative approach.

Current methods of therapeutic delivery to intracerebrally include direct needle bolus injection, implantable port catheter systems, or implantable reservoirs such as the Ommaya reservoir. Administration can be performed by use of an indwelling catheter and a continuous administration means such as a pump, or it can be administered by implantation, e.g., intracerebral implantation of a sustained-release vehicle. More specifically, therapeutic agents can be injected through chronically implanted cannulas or chronically infused with the help of osmotic minipumps. Subcutaneous pumps are available that deliver therapeutic agents through a small tubing to the cerebral ventricles. Highly sophisticated pumps can be refilled through the skin and their delivery rate can be set without surgical intervention. Examples of administration protocols and delivery systems involving a subcutaneous pump device or continuous intracerebroventricular infusion through a totally implanted drug delivery system are those used for the administration of dopamine, dopamine agonists, and cholinergic agonists to Alzheimer's disease patients and animal models for Parkinson's disease, as described by Harbaugh, J. Neural Transm. Suppl. 24:271, 1987; and DeYebenes et. al., Mov. Disord. 2:143, 1987.

The limitation to these systems is that they allow only bolus administration of therapeutic agents and this bolus injection approach may be less than ideal. For example, the Ommaya reservoir includes an injectable reservoir and catheter that accesses the ventricles of the brain. It has been used to sample CSF and administer therapeutic agents directly into the ventricle. With a bolus injection of a therapeutic agent, the concentration of the agent in the patient's ventricle may peak greater than the required therapeutic dose and decrease to below the therapeutic dose before the next administration.

### BRIEF DESCRIPTION OF THE DRAWINGS

These and other aspects will now be described in detail with reference to the following drawings. Generally speaking the figures are not to scale in absolute terms or comparatively but are intended to be illustrative. Also, relative placement of features and elements may be modified for the purpose of illustrative clarity.

FIG. 1A is a cross-sectional exploded view of an implementation of a drug delivery device;

FIG. 1B shows the drug delivery device of FIG. 1A implanted within the brain;

FIG. 1C shows the drug delivery device of FIG. 1A implanted within the brain and the reservoir positioned between the skin and skull;

FIG. 1D is a detailed view of the barbed connector of the device of FIG. 1A;

FIG. 2 shows an interrelated implementation of the drug delivery device of FIG. 1A having a smaller reservoir volume;

FIG. 3 shows high molecular weight monoclonal antibody (mAb) released (%) over time using the device having a reservoir volume of 4.5 mL with no release control element (RCE) or with a high porosity RCE;

FIG. 4 shows mAb released (%) over time using the device having a reservoir volume of 1 mL with no RCE, with high porosity RCE, or with low porosity RCE;

FIG. 5 shows mAb released (%) over time using the device having a reservoir volume of 1 mL with no RCE, with high porosity RCE, or with high porosity RCE having reduced diameter;

FIG. 6 shows low molecular weight fluorescein released (%) over time using the device having a reservoir volume of 1 mL with no RCE, with high porosity RCE, with high porosity RCE having reduced diameter; or with low porosity RCE;

FIG. 7 shows Exponential fitting for mass in device of mAb A over time using the device having a reservoir volume of 1 mL with high porosity RCE, with high porosity RCE having reduced diameter, or low porosity RCE;

FIG. 8 shows Exponential fitting for mass in device of fluorescein over time using the device having a reservoir volume of 1 mL with high porosity RCE, with high porosity RCE having reduced diameter, or low porosity RCE.

### DETAILED DESCRIPTION

Described are therapeutic-delivering devices that provide a controlled and sustained delivery of the ideal therapeutic dose of a therapeutic agent over an extended time. In particular, provided are intraventricular implantable and refillable reservoir devices having a porous metal frit that enables controlled and sustained release of therapeutic agents over time based on the principles of passive diffusion. The devices described herein separate the reservoir from the drug delivery catheter with a porous metal frit that is referred to as the release control element (RCE). The RCE acts as a barrier to diffusion from the reservoir into the catheter. The RCE can be tailored to achieve the desired release profile and can be controlled by changing the porosity of the frit, the size and molecular weight of the drug, and/or the drug concentration in the reservoir. The devices and systems described herein provide new treatment strategies for a variety of clinical issues from delivering chemotherapeutics to brain cancer or monoclonal antibodies, antisense oligonucleotides, gene therapies or cell therapies for neurodegenerative diseases by reducing the frequency of administration and negative side effects from the current bolus administration approaches. The controlled and sustained delivery of the therapeutic agents provide appropriate dosage to avoid underdosing or overdosing. The burden on patients and the healthcare system can be minimized because frequency of injections is minimized reducing the need for clinical visits.

In some variations, one or more of the following can optionally be included in any feasible combination in the above methods, apparatus, devices, and systems. More details of the devices, systems, and methods are set forth in the accompanying drawings and the description below. Other features and advantages will be apparent from the description and drawings.

It should also be appreciated that the devices and systems described herein can be positioned in many locations of the body and need not be implanted specifically as shown in the figures or as described herein. The devices and systems described herein can be used to deliver therapeutic agent(s) for an extended period of time intracranially. The devices and systems can be useful in the treatment of any of a variety of neurodegenerative diseases of the brain including Alzheimer's, stroke, Huntington's, ALS, Angelman syndrome, Parkinson's disease, motor neuron disease, and other diseases of the brain including brain cancer, Batten disease such as late infantile neuronal ceroid lipofuscinosis type 2 (CLN2) also known as tripeptidyl peptidase 1 (TPP1) deficiency, CNS trauma, and other diseases where delivery of a drug directly into the cerebrospinal fluid would be beneficial. Other medical conditions besides these conditions can be treated with the devices and systems described herein. For example, the devices and systems can deliver treatments for inflammation, infection, and cancerous growths. Any number of drug combinations can be delivered using any of the devices and systems described herein.

The materials, compounds, compositions, articles, and methods described herein may be understood more readily by reference to the following detailed description of specific aspects of the disclosed subject matter and the Examples included therein. Before the present materials, compounds, compositions, articles, devices, and methods are disclosed and described, it is to be understood that the aspects described below are not limited to specific methods or specific reagents, as such may vary. It is also to be understood that the terminology used herein is for the purpose of describing particular aspects only and is not intended to be limiting.

### Definitions

Unless defined otherwise, all technical and scientific terms used herein have the same meaning as is commonly understood by one of skill in the art to which the invention(s) belong. All patents, patent applications, published applications and publications, websites and other published materials referred to throughout the entire disclosure herein, unless noted otherwise, are incorporated by reference in their entirety. In the event that there are pluralities of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, relative directional terms such as anterior, posterior, proximal, distal, lateral, medial, sagittal, coronal, transverse, etc. are used throughout this disclosure. Such terminology is for purposes of describing devices and features of the devices and is not intended to be limited. For example, as used herein “proximal” generally means closest to a user implanting a device and farthest from the target location of implantation, while “distal” means farthest from the user implanting a device in a patient and closest to the target location of implantation.

As used herein, a disease or disorder refers to a pathological condition in an organism resulting from, for example, infection or genetic defect, and characterized by identifiable symptoms.

As used herein, treatment means any manner in which the symptoms of a condition, disorder or disease are ameliorated or otherwise beneficially altered. Treatment also encompasses any pharmaceutical use of the devices described and provided herein.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, an effective amount of a compound for treating a particular disease is an amount that is sufficient to ameliorate, or in some manner reduce the symptoms associated with the disease. Such an amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms. Pharmaceutically effective amount, therapeutically effective amount, biologically effective amount and therapeutic amount are used interchangeably herein to refer to an amount of a therapeutic that is sufficient to achieve a desired result, i.e. Therapeutic effect, whether quantitative or qualitative. In particular, a pharmaceutically effective amount, in vivo, is that amount that results in the reduction, delay, or elimination of undesirable effects (such as pathological, clinical, biochemical and the like) in the subject.

As used herein the release rate index encompasses (PA/FL) where P comprises the porosity, A comprises an effective area, F comprises a curve fit parameter corresponding to an effective length and L comprises a length or thickness of the porous structure. The units of the release rate index (RRI) comprise units of mm unless indicated otherwise and can be determined by a person of ordinary skill in the art in accordance with the teachings described hereon.

As used herein, sustained release encompasses release of effective amounts of an active ingredient of a therapeutic agent for an extended period of time. The sustained release may encompass first order release of the active ingredient, zero order release of the active ingredient, or other kinetics of release such as intermediate to zero order and first order, or combinations thereof. The sustained release may encompass controlled release of the therapeutic agent via passive molecular diffusion driven by a concentration gradient across a porous structure.

As used herein, a subject includes any animal for whom diagnosis, screening, monitoring or treatment is contemplated. Animals include mammals such as primates and domesticated animals. An exemplary primate is human. A patient refers to a subject such as a mammal, primate, human, or livestock subject afflicted with a disease condition or for which a disease condition is to be determined or risk of a disease condition is to be determined.

As used herein, a therapeutic agent referred to with a trade name encompasses one or more of the formulation of the therapeutic agent commercially available under the tradename, the active ingredient of the commercially available formulation, the generic name of the active ingredient, or the molecule comprising the active ingredient. As used herein, therapeutic or therapeutic agents are agents that ameliorate the symptoms of a disease or disorder or ameliorate the disease or disorder. Therapeutic agent, therapeutic compound, therapeutic regimen, or chemotherapeutic include conventional drugs and drug therapies, including vaccines, which are known to those skilled in the art and described elsewhere herein. Therapeutic agents include, but are not limited to, moieties that are capable of controlled, sustained release into the body.

As used herein, a composition refers to any mixture. It can be a solution, a suspension, an emulsion, liquid, powder, a paste, aqueous, non-aqueous or any combination of such ingredients.

As used herein, fluid refers to any composition that can flow. Fluids thus encompass compositions that are in the form of semi-solids, pastes, solutions, aqueous mixtures, gels, lotions, creams and other such compositions.

As used herein, a kit is a packaged combination, optionally, including instructions for use of the combination and/or other reactions and components for such use.

The term “oligonucleotide” or “therapeutic oligonucleotide” as used herein is defined as it is generally understood by the skilled person as a molecule comprising two or more covalently linked nucleosides. Such covalently bound nucleosides may also be referred to as nucleic acid molecules, oligonucleotides or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides such as 2′ sugar modified nucleosides.

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides are single stranded. It is understood that single stranded oligonucleotides can form hairpins or intermolecular duplex structures (duplex between two molecules of the same oligonucleotide), as long as the degree of intra or inter self-complementarity is less than 50% across of the full length of the oligonucleotide.

### Treatment Devices

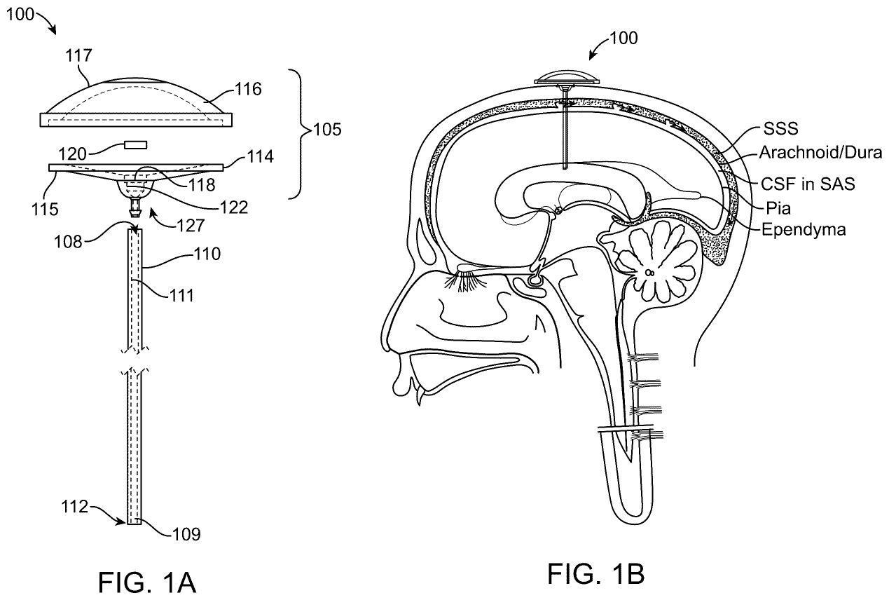

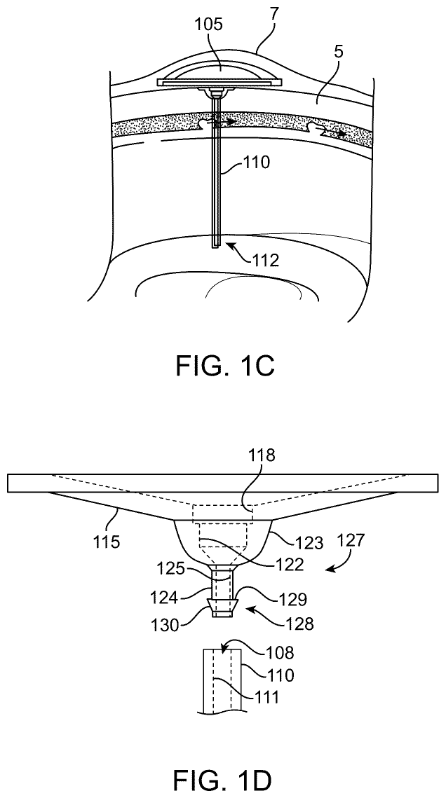

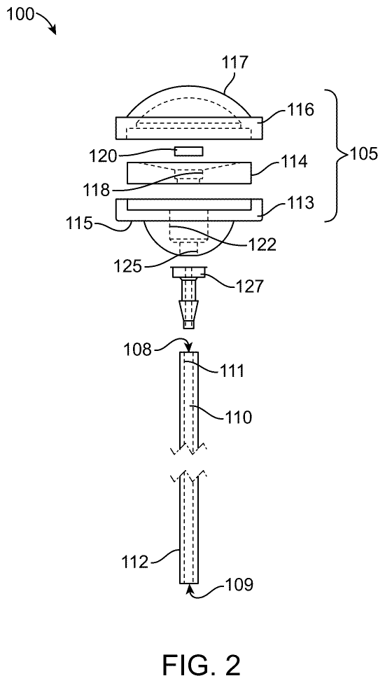

FIGS. 1A and 2 are cross-sectional exploded views of implementations of a drug delivery device 100. Briefly, the device 100 in each implementation includes a container 105 defining a reservoir volume, a catheter 110 configured to be in fluid communication with the reservoir volume of the container 105, and a frit or drug release control element (RCE) 120 that controls the release of therapeutic held within the reservoir volume of the container 105. The RCE 120 can be a porous metal material tailored to adjust the release profile of a therapeutic agent contained within the reservoir based on the surface area, length, and pore size of the RCE 120, which will be described in detail below.

FIGS. 1B-1C are cross-sectional schematic views of the drug delivery device 100 implanted within the brain. The devices described herein can be positioned so that the container 105 remains substantially external of the patient or covered by a layer of skin (i.e., subcutaneous) and the catheter 110 of the device 100 coupled to the container 105 penetrates through the skull until the tip 112 of the catheter 110 is positioned within, adjacent, or in communication the target site. The target site for treatment is preferably intracranial, intracerebral, or intracerebroventricular. The device can be used with a variety of therapeutic agents including one or more chemotherapeutic agents typically delivered in bolus injections for cancer treatment. The therapeutic agent can be peptide, protein, monoclonal antibodies, antisense oligonucleotides, gene therapies, cell therapies, or other small molecules particularly for the treatment of neurological disorders. Therapeutics where drug stability and molecular weight are both important to delivery of antisense oligonucleotides (ASOs) and Cystine-Knot peptides (CKP), disulfide constrained peptides (DCPs) and can be delivered from a small reservoir volume to achieve the therapeutic effect. The devices described herein are particularly useful for delivery of therapeutics that can be delivered from a reservoir volume that is in the 0.5 mL to 5 mL range.

The devices described herein are referred to as drug delivery devices, treatment devices, therapeutic devices, port delivery systems, and the like. It should be appreciated that these terms are used interchangeably herein and are not intended to be limiting to a particular implementation of device over another. For the sake of brevity, explicit descriptions of each of those combinations may be omitted although the various combinations are to be considered herein. Additionally, described herein are different methods for implantation and access of the devices. The various implants can be implanted, filled, refilled etc. according to a variety of different methods and using a variety of different devices and systems. Provided are some representative descriptions of how the various devices may be implanted and accessed, however, for the sake of brevity explicit descriptions of each method with respect to each implant or system may be omitted.

Again with respect to FIG. 1A showing a side cross-sectional view of the device 100, therapeutic agent as described herein can be injected when device 100 is implanted.

The reservoir volume of the container 105 (which may also be referred to herein as a reservoir) may hold an amount of therapeutic agent to be delivered over a period of time. FIG. 1A illustrates the container 105 having a reservoir volume that is larger than a reservoir volume of the container 105 of FIG. 2. For example, the reservoir volume can be between 0.5 mL up to about 5.0 mL. The overall size of the container 105 can be selected to be relatively low profile so that the container 105 can be implanted between the skull and the skin. A lower surface 115 of the container 105 can be placed against the skull bone 5 and an upper surface 117 of the container 105 can be covered by the overlying skin 7 (see FIG. 1C). Thus, a majority of the container 105 is substantially implanted inside the patient, but external to the skull 5. The diameter of the container 105 can be between about 10 mm to about 45 mm. The maximum height of the container 105 between the lower surface 115 configured to sit against the skull 5 to the upper surface 117 can be between about 3 mm and about 15 mm.

The container 105 can be oval, elliptical, or circular in perimetrical shape. The lower surface 115 can be flat or have a curved profile that conforms substantially to the shape of the skull 5 at a particular location. The upper surface 117 can be substantially convex spherical or toric. In some implementations, the container 105 has a rounded profile avoiding sharp or square edges that could cause discomfort to a patient upon implantation. The material of the container 105 can be relatively rigid silicone or other polymeric material such that the reservoir volume of the container 105 regardless the fill status remains substantially constant. Alternatively or in combination, the material of the container 105 can be relatively soft or elastomeric, for example, so that as therapeutic agent 10 is removed from or introduced into the container 105 the contour of the container 105 changes. A soft material of the container 105 can be combined with a rigid material, for example, to provide support.

The container 105 can be formed by a lower base 114 including the lower surface 115 and an upper cap 116 including the upper surface 117. The base 114 and cap 116 together define the reservoir volume of the container 105. At least a portion of the cap 116 including the entire cap 116 can be formed of a material that is configured to be penetrated with a sharp object such as a needle for injection of the therapeutic agent into the reservoir volume. The cap 116 can be coupled (such as by epoxy) and sealed with the base 114 to create the liquid-tight reservoir volume of the container 105. The base 114 can define the outlet from the container 105.

The internal surface of the base 114 can include a seat 118 for scaling with the RCE 120. The seat 118 in FIGS. 1A and 2 is located in the bottom of the reservoir 105 away from the penetrable cap 116. The RCE, which is generally porous titanium or stainless steel, fits tightly within the seat 118 of the base 114, such as by a press fitting, heat swaging, epoxy, into the impermeable structure of the container. The tight fit ensures diffusion occurs through the RCE as opposed to around the RCE.

Below the seat 118 is a diffusion chamber 122 located within a projection 124 from the lower surface 115 of the base 114. The diffusion chamber 122 can have an inner diameter that is less than the inner diameter of the container 105 located above it and greater than an inner diameter of an outlet channel 125 located below it and extending through a barbed connector 127. The diffusion chamber 122 tapers along a length to the smaller inner diameter of the outlet channel 125. The inner diameter of the outlet channel 125 can have a smaller inner diameter than the diffusion chamber 122 and also a smaller inner diameter than the lumen 111 of the catheter 110. The size of the diffusion chamber 122 located below the location of the RCE 120 and above the outlet channel 125 allows the entire surface area of the RCE 120 seated above it to be used before narrowing to the outlet channel 125 to connect with the lumen 111 of the catheter 110.

As best shown in FIG. 1D, the barbed connector 127 projects from the lower surface 115 of the base 114 and can similarly change in outer diameter from an upper end to a lower end. The upper region 123 of the barbed connector 127 defining the diffusion chamber 122 can have a first outer diameter. The first outer diameter can be less than the outer diameter of the container 105 and greater than an outer diameter of the catheter 110. The upper region 123 of the barbed connector 127 can taper along a length to a lower region 124. The lower region 124 of the barbed connector 127 can have a smaller outer diameter than the upper region 123 and also a smaller outer diameter than the outer diameter of the catheter 110. The lower region 124 of the barbed connector 127 can incorporate a barb 128 or other surface feature near its distal end that ensures the connection between the container 105 and the catheter 110 is snug and will not be unintentionally dislodged. The barb 128 can have a slightly larger outer diameter than the inner diameter of the catheter 110 so that as the barbed connector 127 is inserted through the proximal opening 108 of the catheter 110 the material of the catheter 110 deforms slightly so as to accommodate the larger outer diameter by interference fit. The planar upper surface 129 and square edge of the barb 128 is harder to withdraw from the lumen 111 than the tapered leading surface 130 of the barb 128 is to insert within the lumen 111. Upon connecting the base 114 to the catheter 110 the outlet channel 125 is placed in fluid communication with the lumen 111 of the catheter 110 with the RCE 120 controlling release of therapeutic into the lumen 111 from the reservoir volume.

In the embodiment shown in FIG. 2, the base 114 can be epoxied to the cap 116 creating a puncturable reservoir to contain the therapeutic agent. The barbed connector 127 can be epoxied to a bottom component 113 and then with subassembly is epoxied to the base 114. This embodiment can create a secondary fluid space to allow diffusion through the entire surface of the RCE 120. The barbed connector 127 has more freedom and flexibility because it is not directly attached to the base 114 as in the embodiment shown in FIG. 1A. The implant can also have a smaller overall size so as to be less invasive.

The elongate catheter 110 is coupled to a lower end of the container 105 as shown in FIGS. 1A and 2. The catheter 110 can be centered relative to the lower end of the container 105 or can be eccentric. The lumen 111 of the catheter 110 extends from the proximal opening 108 into the catheter 110 to at least one distal opening 109 near the tip 112 of the catheter 110. The catheter 110 has a length sized so as to extend from outside the skull 5 to a target location to release the therapeutic agent into a region of the brain, preferably within the dural sinuses or ventricles of the brain. The length can be sized within a range, for example, from about 10 cm up to about 20 cm. The length can be less than about 15 cm to as short as about 10 cm, about 5 cm, about 3 cm, or a length that is sufficient to extend through a craniotomy burr hole into, for example, a ventricle of the brain or within one or more of the dural sinuses. In an implementation, the catheter length can be between 3 cm to 5 cm, up to about 15 cm. The catheter 110 can also be provided having a length that is greater than these ranges (e.g., between about 30 cm-50 cm) and then cut to size by a user at the time of implantation to achieve a final preferred length. The catheter 110 can be manufactured to have a single size greater than a length needed to reach a target site upon implantation so that any of a variety of target locations can be treated with the same modular system. The device can be provided with the catheter separated from the barb so that the proximal end of the catheter can be cut to size and then pressed onto the barb prior to implantation. Cutting the proximal end to adjust the size of the catheter 110 avoids impacting the distal tip 112, which can have specialized distal opening(s) 109 to deliver the therapeutic. The catheter 110 can have one or more markings on its outer surface near the proximal end to guide trimming to size and to indicate a length to the distal tip 112 dependent on where the proximal end is cut.

The cross sectional outer diameter of the catheter 110 can be sized to decrease the invasiveness of device 100, and may comprise an outer diameter of no more than about 5 mm, preferably about 1 mm to about 4 mm. The inner diameter of the catheter 110 can be at least about 0.5 mm, preferably about 0.5 mm to about 3 mm.

The catheter distal tip 112 can be shaped to include one or more outlets including the distal opening 109. The distal tip 112 of the catheter 110 can have one or more outlets extending through a wall of the catheter 110. The plurality of outlets can be positioned through the wall can vary in size from about 0.0008 inch (0.02 mm) to about 0.08 inch (2.03 mm), preferably between about 0.008″ (0.2 mm) to about 0.05″ (1.2 mm). The outlets can vary in shape and number as well, including one or more rows of about 2 to about 20 holes having circular, oval, or other shape. The outlets can be spaced angularly at about 90 degrees from each other so as to occur circumferentially around the distal tip 112. The outlets can be spaced apart circumferentially by about 45 degrees, 90 degrees, 120 degree, or 180 degrees.

The catheter 110 can be formed of silicone or polyurethane or a combination of materials. One or more regions of the catheter 110 can be reinforced such as with a tube, cut tube, coil, braid, or other reinforcement structure. The reinforcement structure can be a metal material or a plastic material having a greater durometer than the remaining portion of the catheter 110. The barb 128 and base 114 can be made of a more rigid plastic than the proximal end region of the catheter 110 including polysulfone or polymethyl methacrylate or other suitable plastic. The more rigid material of the barb 128 and/or base 114 allows for these portions of the device to deform and more sufficiently connect with the softer catheter material.

The catheter 110 is preferably kept straight between its proximal and distal ends without any turns between the container 105 and the site being treated. Thus, the container 105 upon implantation is generally positioned above the distal tip 112 of the catheter 110 positioned within the target site. The vertical orientation of the straight catheter 110 ensures delivery of the therapeutic from the container 105, through the lumen, and out the distal tip 112 into the target site.

The RCE 120 can be located relative to the catheter 110 so as to release therapeutic agent for an extended period from the container 105. The frit can be tailored to adjust the release profile of the therapeutic agent. Specifically, parameters including surface area of the RCE, length, tortuosity, and pore size of the RCE can all be tailored to achieve the release profile desired. The therapeutic agent size, molecular weight, and concentration also influences the release profile. The length of the catheter contributes to the tortuosity parameter of the release rate. Thus, the RCE is relatively porous compared to a drug delivery device having a substantially shorter catheter or cannula.

In many embodiments, the RCE 120 comprises a porosity, a thickness, a channel parameter and a surface area configured to release therapeutic amounts for the extended period. The porosity may comprise a value within a range from about 1% to about 70%, preferably 15%-40%. The porosity may comprise a value within a range from about 3% to about 30%. The porosity may comprise a value within a range from about 5% to about 10%. The porosity may comprise a value within a range is from about 10% to about 25%. The porosity may comprise a value within a range is from about 10% to about 20%.

In many embodiments, the channel parameter comprises a fit parameter corresponding to the tortuosity of the channels.

In many embodiments, the channel parameter comprises a fit parameter corresponding to an effective length of interconnecting channels extending from a first side of the RCE 120 to a second side of the RCE 120. The effective length of the interconnecting channels may correspond to at least about 2 times a thickness of the RCE 120. The effective length of the interconnecting channels may correspond to at least about 5 times a thickness of the RCE 120. The channel parameter can be between 1 and 10, preferably between 2 and 5.

In many embodiments, the rate of release of the at least one therapeutic agent corresponds to a ratio of the porosity to the channel parameter, and the ratio of the porosity to the channel parameter is less than about 0.5 such that the RCE 120 releases the at least one therapeutic agent for the extended period. The ratio of the porosity to the channel parameter can be less than about 0.2 such that the RCE 120 releases the at least one therapeutic agent for the extended period. The ratio of the porosity to the channel parameter can be less than about 0.1 such that the RCE 120 releases the at least one therapeutic agent for the extended period. The ratio of the porosity to the channel parameter can be less than about 0.05 such that the RCE 120 releases the at least one therapeutic agent for the extended period. The ratio of the porosity to the channel parameter can be between 0.001 to 0.7, preferably between 0.03 and 0.2.

In many embodiments, the channel parameter comprises a value of at least about 1. The value of the channel parameter may comprise at least about 2. The channel parameter may comprise a value of at least about 5. The channel parameter may comprise a value of at least about 10.

In many embodiments, RCE 120 comprises a release rate index (RRI) determined with a ratio of the porosity times a cross-sectional area of the RCE 120 divided by the channel parameter times a thickness of the RCE 120, the thickness extending across the cross sectional area. The release rate index range can be 0.01-20 although the RRI range can change depending on molecule and desired time of release. The RCE 120 may comprise a release rate index of no more than about 5.0 mm. The RCE 120 may comprise a release rate index of no more than about 2 mm. The RCE 120 may comprise a release rate index of no more than about 1.2 mm. The RCE 120 may comprise a release rate index of no more than about 0.2 mm. The RCE 120 may comprise a release rate index of no more than about 0.1 mm. The RCE 120 may comprise a release rate index of no more than about 0.05 mm.

The devices 100 described herein can include one or more RCEs 120. The RCE 120 (also referred to herein as a drug release mechanism, drug release element, release control element, porous structure, or frit) as described herein can be positioned adjacent and/or within an outlet channel 125 from the device 100 such that the RCE 120 can control or regulate the delivery of the one or more therapeutic agents from the container 105 through the outlet channel 125. The contents of the container 105 can be delivered according to slow diffusion rather than expelled as a fluid stream or a bolus. In some implementations, the RCE 120 can be disposed within a region of the container 105 that communicates with the catheter 110. In some implementations, the RCE 120 can be a covering or lining having a particular porosity to the substance to be delivered and can be used to provide a particular rate of release of the substance.

The RCE 120 can include, but is not limited to a wicking material, permeable silicone, packed bed, small porous structure or a porous frit, multiple porous coatings, nanocoatings, rate-limiting membranes, matrix material, a sintered porous frit, a permeable membrane, a semi-permeable membrane, a capillary tube or a tortuous channel, nano-structures, nano-channels, sintered nanoparticles and the like. The RCE 120 can have a porosity, a cross-sectional area, and a thickness to release the one or more therapeutic agents for an extended time from the container 105. The porous material of the RCE 120 can have a porosity corresponding to a fraction of void space formed by channels extending through or between the material. The void space formed can be between about 1% to about 70%, between about 5% to about 10%, between about 10% to about 25%, or between about 15% to about 20%, or any other fraction of void space.

The osmolarity and tonicity of the cerebrospinal fluid can be within a range from about 290.5 mOsm/L to about 291.5 mOsm/L (see Akaishi et. al., Neural Regen Res, 2020 May 15 (5): 944-947). For example, a commercially available formulation of a therapeutic agent to be delivered may be diluted so as to have a formulation having an osmolarity and tonicity substantially similar to the osmolarity and tonicity of the CSF, for example about 290 mOsm/L. While the therapeutic agent may have an osmolarity and tonicity substantially similar to the CSF, the therapeutic agent may have a hyper osmotic (hypertonic) solution relative to the CSF or a hypo osmotic (hypotonic) solution relative to the CSF. A person or ordinary skill in the art can conduct experiments based on the teachings described herein so as to determine empirically the formulation and osmolarity of the therapeutic agent to provide release of therapeutic agent for an extended time.

The RCE 120 has a first side facing the reservoir volume of the container 105 and a second side facing the proximal opening 108 into the catheter lumen 111. The first side has a first area as described herein and the second side has a second area. The RCE 120 has a thickness between the first and second sides and a diameter. The RCE 120 has a release rate index, which will be described in detail below. The RCE 120 includes a fixed tortuous, porous material such as a sintered metal, a sintered glass or a sintered polymer with a defined porosity and tortuosity that controls the rate of delivery of at least one therapeutic agent to the target site. The rigid RCE 120 provides certain advantages over capillary tubes, erodible polymers and membranes as a mechanism for controlling the release of a therapeutic agent or agents from the therapeutic device. These advantages include the ability of the rigid RCE 120 to be simpler and more cost effective manufacture, flushability for cleaning or declogging either prior to or after implantation, high efficiency depth filtration of microorganisms provided by the labyrinths of irregular paths within the structure and greater robustness due to greater hardness and thickness of the structure compared to a membrane or erodible polymer matrix. In implementations, drug can be injected into the reservoir, flushed, and then refilled with drug. The flush can push a first bolus of drug deeper into the brain tissues and the second fill of drug used for extended duration release. Additionally, when the rigid RCE 120 is manufactured from a sintered metal, ceramic, glass or certain plastics, it can be subjected to sterilization and cleaning procedures, such as heat or radiation based sterilization and depyrogenation, that might damage polymer and other membranes.

In certain embodiments, as illustrated in Example 1, the rigid RCE 120 may be configured to provide a therapeutically effective, concentration of the therapeutic agent in the CSF for at least 1 month, at least 2 months, at least 3 months up to about 6 months, 12 months, 24 months, or up to about 36 months. The time of treatment can vary depending on the diffusivity, molecular size/weight, concentration, half-life, stability, and volume of therapeutic agent as well as the parameters of the RCE. The release profile can vary from as short as a few days to as long as several months to years depending on the specific application, drug potency, as well as the features of the device and physical properties of the RCE. This release profile provided by certain configurations of the rigid RCE 120 enables a smaller device, which is preferred in intracerebroventricular treatments where larger devices may impact device invasiveness, patient comfort and potentially lead to problems with infection or leakage.

The RCE 120 may include a plurality of interconnecting channels formed between sintered grains of a material such as titanium or stainless steel. The interconnected grains of material define space through the RCE 120 that the therapeutic agent can pass through to reach the second side of the RCE 120 from the first side of the RCE 120. The channels may extend around the sintered grains of material, such that the channels comprise interconnecting channels extending through the porous material.

The rigid RCE 120 can have a hardness parameter within a range from about 160 Vickers to about 500 Vickers. In some embodiments the rigid RCE 120 is formed from sintered stainless steel. The material of the RCE may have a hardness parameter within a range from about 200 Vickers to about 240 Vickers. In some embodiments it is preferred to inhibit ejection of the therapeutic agent through the RCE 120 during filling or refilling the container 105 of the therapeutic device 100 with a fluid. In these embodiments the channels of the rigid RCE 120 have a resistance to flow of an injected solution or suspension such that ejection of the solution or suspension through the RCE 120 is substantially inhibited when the solution or suspension is injected into the container 105 of the therapeutic device 100.

The container 105 and the RCE 120 can be configured to release therapeutic amounts of the therapeutic agent in many ways. The container 105 and the RCE 120 can be configured to release therapeutic amounts of the therapeutic agent corresponding to a concentration of at least about 1 mg/mL up to about 300 mg/mL for at least about a plurality of days up to about 12 months. The therapeutic agent may be at least a fragment of an antibody and a molecular weight of at least about 150 Daltons. For example, the therapeutic agent may be about 8,000-200,000 Daltons. Alternatively or in combination, the therapeutic agent may be a small molecule drug suitable for sustained release having a molecular weight of about 100 Daltons to about 10,000 Daltons. The therapeutic agent may be one or more different antisense oligonucleotides, CKPs, various immunotherapies such as monoclonal antibodies, as well as thrombolytics, protease inhibitors and others useful for treating various neurodegenerative disorders.

The reservoir and the RCE 120 may be configured to release therapeutic amounts of the therapeutic agent corresponding to a concentration of at least about 1 mg/mL, 100 mg/mL, 200 mg/mL, or 300 mg/mL for an extended period of a few days, 1 month, 2 months, 3 months, 6 months, 12 months, 24 months, up to about 36 months and anywhere in between.

The rigid RCE 120 may include a composite porous material that can readily be formed in or into a wide range of different shapes and configurations. For example, the porous material can be a composite of a metal, aerogel or ceramic foam (i.e., a reticulated inter-cellular structure in which the interior cells are interconnected to provide a multiplicity of pores passing through the volume of the structure, the walls of the cells themselves being substantially continuous and non-porous, and the volume of the cells relative to that of the material forming the cell walls being such that the overall density of the intercellular structure is less than about 30 percent theoretical density) the through pores of which are impregnated with a sintered powder or aerogel. The thickness, density, porosity and porous characteristics of the final composite porous material can be varied to conform with the desired release of the therapeutic agent.

The release rate of therapeutic agent through a porous body, such as a sintered porous metal structure or a porous glass structure, may be described by diffusion of the of the therapeutic agent within the RCE 120 with the channel parameter, and with an effective diffusion coefficient equal to the diffusion coefficient of the therapeutic agent in the liquid that fills the reservoir multiplied by the Porosity and a Channel Parameter of the porous body:

$$Release Rate = ( D P / F ) A ( {C}_{R} - Cp ) / L ,$$

where:
C_{R}=Concentration in reservoir
Cₚ=Concentration outside of the reservoir or at the target site
D=Diffusion coefficient of the therapeutic agent in the reservoir solution
P=Porosity of porous structure
F-Channel parameter that may correspond to a tortuosity parameter of channels of
porous structure
A=Area of porous structure
L=Thickness (length) of porous structure

$$Cumulative Release = 1 - cR / cR 0 = 1 - exp ( ( - D PA / FL {V}_{R} ) t ) ,$$

where
t=time, Vr=reservoir volume

The release rate index can (hereinafter RRI) be used to determine release of the therapeutic agent. The RRI may be defined as (PA/FL), and the RRI values herein will have units of mm unless otherwise indicated. Many of the RCEs 120 used in the therapeutic delivery devices described here have an RRI of between 0.01 and 20, although the RRI range can vary depending on the molecule and desired time of release and application.

The channel parameter can correspond to an elongation of the path of the therapeutic agent released through the RCE 120. The RCE 120 may include many interconnecting channels, and the channel parameter can correspond to an effective length that the therapeutic agent travels along the interconnecting channels of the RCE 120 from the reservoir side to the patient side when released. The channel parameter multiplied by the thickness (length) of the RCE 120 can determine the effective length that the therapeutic agent travels along the interconnecting channels from the reservoir side to the patient side. For example, the channel parameter (F) of about 1.5 corresponds to interconnecting channels that provide an effective increase in length traveled by the therapeutic agent of about 50%, and for a 1 mm thick RCE 120 the effective length that the therapeutic agent travels along the interconnecting channels from the reservoir side to the patient side corresponds to about 1.5 mm. The channel parameter (F) of at least about 2 corresponds to interconnecting channels that provide an effective increase in length traveled by the therapeutic agent of about 100%, and for a 1 mm thick RCE 120 the effective length that the therapeutic agent travels along the interconnecting channels from the reservoir side to the patient side corresponds to at least about 2.0 mm. As the RCE 120 includes many interconnecting channels that provide many alternative paths for release of the therapeutic agent, blockage of some of the channels provides no substantial change in the effective path length through the RCE 120 as the alternative interconnecting channels are available, such that the rate of diffusion through the RCE 120 and the release of the therapeutic agent are substantially maintained when some of the channels are blocked.

If the reservoir solution is aqueous or has a viscosity similar to water, the value for the diffusion coefficient of the therapeutic agent (TA) in water at the temperature of interest may be used. The following equation can be used to estimate the diffusion coefficient at 37° C. from the measured value of D_{BSA.20C}=6.1 e-7 cm²/s for bovine serum albumin in water at 20° C. (Molokhia et al, Exp Eye Res 2008):

$${D}_{TA , 37 C} = {D}_{BSA , 20 C} ( {η}_{20 C} / {η}_{37 C} ) {( {MW}_{BSA} / {MW}_{TA} )}^{1 / 3}$$

where
MW refers to the molecular weight of either BSA or the test compound and η is the viscosity of water. Table 1 below lists diffusion coefficients of proteins of interest.

TABLE 1

| Compound | MW | Temp C. | Diff. Coeff (cm²/s) |
|---|---|---|---|
| BSA | 69,000 | 20 | 6.1E−07 |
| BSA | 69,000 | 20 | 6.1E−07 |
| mAB | 150,000 | 20 | 6.2E−07 |
| Fluorescein | 332 | 20 | 6.9E−06 |

Small molecules have a diffusion coefficient similar to fluorescein (MW=330, D=4.8 to 6 e-6 cm²/s from Stay, M S et al. Pharm Res 2003, 20 (1), pp. 96-102). For example, the small molecule may comprise a glucocorticoid such as triamcinolone acetonide having a molecular weight of about 435.

The RCE 120 have a porosity, a thickness, a channel parameter and a surface area configured to release therapeutic amounts for the extended period. The porous material may have a porosity corresponding to the fraction of void space of the channels extending within the material. The porosity includes a value within a range from about 1% to about 70%, preferably 15%-40%. Porosity can be determined from the weight and macroscopic volume or can be measured via nitrogen gas adsorption.

The RCE 120 may include a plurality of porous structures, and the area used in the above equation may be the combined area of the plurality of porous structures.

The channel parameter may include a fit parameter corresponding to the tortuosity of the channels. For a known porosity, surface area and thickness of the surface parameter, the curve fit parameter F, which may correspond to tortuosity of the channels can be determined based on experimental measurements. The parameter PA/FL can be used to determine the desired sustained release profile, and the values of P, A, F and L determined. The rate of release of the therapeutic agent corresponds to a ratio of the porosity to the channel parameter, and the ratio of the porosity to the channel parameter can be less than about 0.7 such that the RCE 120 releases the therapeutic agent for the extended period. For example, the ratio of the porosity to the channel parameter is less than about 0.1 or for example less than about 0.2 such that the RCE 120 releases the therapeutic agent for the extended period. The channel parameter may comprise a value of at least about 1, such as at least about 1.2. For example, the value of the channel parameter may comprise at least about 1.5, for example at least about 2, and may comprise at least about 5. The channel parameter can be within a range from about 1.1 to about 10, for example within a range from about 2 to about 5. A person of ordinary skill in the art can conduct experiments based on the teachings described herein to determine empirically the channel parameter to release the therapeutic agent for an intended release rate profile.

The area in the model originates from the description of mass transported in units of flux; i.e., rate of mass transfer per unit area. For simple geometries, such as a porous disc mounted in an impermeable sleeve of equal thickness, the area corresponds to one face of the disc and the thickness, L, is the thickness of the disc. For more complex geometries, such as a porous body in the shape of a truncated cone, the effective area is a value in between the area where therapeutic agent enters the porous body and the area where therapeutic agent exits the porous body. The porosity of the RCE 120 can be high enough that the length of the catheter does not impact or minimally impacts the release rate from the device. The rate of drug release is controlled by the porosity of the RCE 120, the size of the drug molecule being delivered, and/or the concentration of the drug in the reservoir. The reservoir is preferably positioned directly over the catheter and no tortuosity present in the catheter from the reservoir to the target treatment area. This vertical orientation between the distal outlet from the catheter and the inlet from the reservoir is preferred to ensure good drug diffusion to the target treatment area.

A model can be derived to describe the release rate as a function of time by relating the change of concentration in the reservoir to the release rate described above. This model assumes a solution of therapeutic agent where the concentration in the reservoir is uniform. In addition, the concentration in the receiving fluid is considered negligible (Cₚ=0). Solving the differential equation and rearrangement yields the following equations describing the concentration in the reservoir as a function of time, t, and volume of the reservoir, V_{R}, for release of a therapeutic agent from a solution in a reservoir though a porous structure.

$${C}_{R} = {C}_{RO} exp ( ( - D PA / FL {V}_{R} ) t )$$

and Cumulative Release=1−C_{R}/C_{RO}

When the reservoir contains a suspension, the concentration in the reservoir, C_{R}, is the dissolved concentration in equilibrium with the solid (i.e., the solubility of the therapeutic agent). In this case, the concentration in the reservoir is constant with time, the release rate is zero order, and the cumulative release increases linearly with time until the time when the solid is exhausted.

Therapeutic concentrations for many therapeutic agents may be determined experimentally by measuring concentrations in the target site that elicit a therapeutic effect. Therefore, there is value in extending predictions of release rates to predictions of concentrations in the target site. The majority of cerebrospinal fluid (CSF) is produced by the choroid plexus in the ventricles, circulates through the ventricles, the cisterns, and the subarachnoid space to be absorbed into the blood by the arachnoid villi. The CSF circulation includes not only a directed flow of CSF, but in addition a pulsatile to and fro movement throughout the entire brain with local fluid exchange between blood, interstitial fluid, and CSF. The CSF has a physiologic volume of about 150-200 ml in adults with a daily turnover of about 500 ml. For devices with extended release, the concentration in the cerebrospinal fluid (CSF) changes slowly with time. In this situation, a model can be derived from a mass balance equating the release rate from the device (described by equations above) with the elimination rate from the CSF, k, Cₚ, Vᵥ. Rearrangement yields the following equation for the concentration in the CSF:

$${C}_{p} = Release rate from device / k {V}_{V} .$$

Since the release rate from a device with a solution of therapeutic agent decreases exponentially with time, the concentration in the CSF decreases exponentially with the same rate constant. In other words, CSF concentration decreases with a rate constant equal to D PA/FL V_{R} and, hence, is dependent on the properties of the porous structure and the volume of the reservoir.

Since the release rate is zero order from a device with a suspension of therapeutic agent, the CSF concentration will also be time-independent. The release rate will depend on the properties of the porous structure via the ratio, PA/FL, but will be independent of the volume of the reservoir until the time at which the drug is exhausted.

The channels of the rigid porous structure can be sized in many ways to release the intended therapeutic agent. For example, the channels of the rigid porous structure can be sized to pass therapeutic agent comprising molecules having a molecular weight of at least about 100 Daltons or for example, at least about 200 k Daltons. The channels of the rigid porous structure can be sized to pass therapeutic agent comprising molecules comprising a cross-sectional size of no more than about 10 nm. The channels of the rigid porous structure comprise interconnecting channels configured to pass the therapeutic agent among the interconnecting channels. The rigid porous structure comprises grains of rigid material and wherein the interconnecting channels extend at least partially around the grains of rigid material to pass the therapeutic agent through the porous material. The grains of rigid material can be coupled together at a loci of attachment and wherein the interconnecting channels extend at least partially around the loci of attachment.

The porous structure comprises a sintered material. The sintered material may comprise grains of material in which the grains comprise an average size of no more than about 20 μm. For example, the sintered material may comprise grains of material in which the grains comprise an average size of no more than about 10 μm, an average size of no more than about 5 μm, or an average size of no more than about 1 μm. The channels are sized to pass therapeutic quantities of the therapeutic agent through the sintered material for the extended time based on the grain size of the sintered material and processing parameters such as compaction force and time and temperature in the furnace. The channels can be sized to inhibit penetration of microbes including bacterial and fungal spores through the sintered material.

The sintered material comprises a wettable material to inhibit bubbles within the channels of the material.

The sintered material comprises at least one of a metal, a ceramic, a glass or a plastic. The sintered material may comprise a sintered composite material, and the composite material comprises two or more of the metal, the ceramic, the glass or the plastic. The metal comprises at least one of Ni, Ti, nitinol, stainless steel including alloys such as 304, 304L, 316 or 316L, cobalt chrome, elgiloy, hastelloy, c-276 alloy or Nickel 200 alloy. The sintered material may comprise a ceramic. The sintered material may comprise a glass. The plastic may comprise a wettable coating to inhibit bubble formation in the channels, and the plastic may comprise at least one of polyether ether ketone (PEEK), polyethylene, polypropylene, polyimide, polystyrene, polycarbonate, polyacrylate, polymethacrylate, or polyamide. Any material capable of forming a continuous porous channel system and that is biocompatible is also considered herein for the porous structure.

The channel parameter and effective length from the first side to the second side can be configured in many ways. The channel parameter can be greater than 1 and within a range from about 1 to about 10, preferably between about 2 to about 5.0, such that the effective length is within a range about 2 to 5.0 times the thickness, although the channel parameter may be greater than 5, for example within a range from about 1.2 to 10. For example, the channel parameter can be from about 1.3 to about 2.0, such that the effective length is about 1.3 to 2.0 times the thickness. For example, experimental testing has shown the channel parameter can be from about 1.4 to about 1.8, such that the effective length is about 1.4 to 1.8 times the thickness, for example about 1.6 times the thickness. The channel parameter may comprise a value of at least about 10. These values correspond to the paths of the channels around the sintered grains of material, and may correspond, for example, to the paths of channels around packed beads of material.

The rigid porous structure 120 can be shaped and molded in many ways for example with tubular shapes, conical shapes, discs and hemispherical shapes. The rigid porous structure may be a molded rigid porous structure. The molded rigid porous structure 120 may be at least one of a disk, a helix or a tube coupled to the reservoir and configured to release the therapeutic agent for the extended period.

The porous structure 120 may include a plurality of elongate nano-channels extending from a first side of the porous structure to a second side of the porous structure. The porous structure 120 may be a rigid material having the holes formed thereon, and the holes may comprise a maximum dimension across such as a diameter. The diameter of the nano-channels may comprise a dimension across, for example from about 10 nm across, to about 1000 nm across, or larger. The channels may be formed with etching of the material, for example lithographic etching of the material. The channels may comprise substantially straight channels such that the channel parameter F comprises about 1, and the parameters area A, and thickness or length L correspond to the combined cross-sectional area of the channels and the thickness or length of the porous structure.

Mathematical modeling can be potentially used to predict how the reservoir container 110 with the incorporated RCE 120 behaves in a molecule and concentration specific manner. Using Fick's Law of Diffusion and mass balance equations, the following equation (Eq. 1) can be derived:

$$\frac{{dm}_{dev}}{dt} = - D * RRI * {C}_{dev , o} * {e}^{( \frac{- D * RRI}{{V}_{dev}} * t )} . \qquad Eq . 1$$

D is the diffusion coefficient of the therapeutic agent, RRI is a RCE-defining property defined in more detail below, C_{dev,o} is the initial concentration in the device, V_{dev} is the device's volume, t is time, and m_{dev} is the mass in the device. The following assumptions are made: (1) no drug is lost by any other means than diffusion, release of agent is driven by concentration gradient, catheter concentration is much less than device concentration, and the rate limiting step is diffusion across the RCE.

The RRI as described herein can be determined for the RCE 120 having the plurality of elongate nano-channels that extend substantially straight through the RCE 120. The RRI can be determined with the equation (Eq. 2) RRI=(P*A)/(F*L) as described herein, where P=RCE porosity, A=RCE surface area, F=RCE tortuosity channel parameter, and L=length (thickness) of RCE 120. The channel parameter F corresponds to 1 for straight channels, and the porosity P corresponds to the percentage of the surface area of the RCE 120 having the substantially straight nano-channels. For example, a flat plate having a surface area of 1 mm², a thickness of 0.5 mm, and holes over 10% of the surface area, the corresponding RRI is determined as (0.1*1)/(1*0.5)−0.2. Based on the teachings described herein a person of ordinary skill in the art can determine the surface area A and thickness L of the RCE 120, percentage of surface area of the nano-channels, so as to provide an appropriate RRI for the therapeutic agent and reservoir volume of device 100 as described herein.

Integrating Eq. 1 with initial conditions of mass initial is Mₒ and the mass at time infinity is 0 yield the following formula for mass in the device over time (Eq. 3):

$${M}_{dev} ( t ) = {M}_{o} {e}^{( \frac{- D * RRI}{{V}_{dev}} * t )}$$

### EXPERIMENTAL

### Example 1

Tests were conducted to determine feasibility of different RCEs to influence the release of a drug from the reservoir devices described herein into the CNS. Tests were conducted using reservoir devices having a 4.5 mL volume size and 1 mL volume size. The catheter length for each reservoir size was 11 cm and the administration was intracerebroventricular where the reservoir was positioned above the target treatment site so as to achieve vertical or near vertical orientation between the reservoir and the target such that the delivery of the drug may be assisted by gravity. Table 2 below shows the device configurations tested. The catheter can be ⅛″ outer diameter (3.175 mm OD) and 1/16″ inner diameter (1.5875 mm ID).

TABLE 2
Device Configurations Tested

| | | Device | Catheter | |
|---|---|---|---|---|
| | | Volume | Length | |
| Device Type | Administration | (mL) | (cm) | Orientation |
| Reservoir and Catheter | ICV | 4.5 | 11 | Vertical |
| Reservoir and Catheter | ICV | 1 | 11 | Vertical |

The reservoirs of the devices were filled with a monoclonal antibody (mAb A) as a model large molecule therapeutic agent or fluorescein as a model small molecule therapeutic agent. Table 3 below shows the list of molecules tested for sustained delivery. Antisense oligonucleotides are generally in the 3000-7000 m.w. size range and would benefit from sustained release.

TABLE 3
List of Molecules tested for sustained delivery

| Molecule Type | Molecular Weight (Da) |
|---|---|
| Large molecule, Monoclonal antibody (mAb A) | Approx. 150,000 |
| Small molecule, Fluorescein | 332.31 |

The RCEs varied in diameter, thickness, porosity, and micron grade. Some devices contained high porosity RCEs (types 334, 011, 012) and others contained lower porosity RCEs (types 014, 627). Still other devices included no RCEs. Table 4 below shows the different RCEs that were tested.

TABLE 4
List of RCEs

| RCE ID | Diameter (in) | Thickness (in) | Porosity | Micron Grade |
|---|---|---|---|---|
| 334 | 0.197 | 0.062 | High | 5.0 |
| 011 | 0.188 | 0.062 | High | 2.0 |
| 012 | 0.125 | 0.062 | High | 2.0 |
| 014 | 0.188 | 0.062 | Low | 0.5 |
| 627 | 0.188 | 0.062 | Low | 0.2 |

The devices were filled with the mAb A at about 200 mg/mL and fill volume was calculated from the difference in mass of the unfilled and filled device using the density of the mAb A solution. Each device's initial internal mass was calculated based on the known volume and concentration. The catheters were filled with phosphate buffer saline (PBS) and attached to the filled reservoir. The devices were oriented to release the therapeutic into a sink of 50 mL PBS. The sink was gently agitated at 100 rpm on an orbital shaker at 37° C. to simulate body movement. Over time, the sink was sampled and the mAb concentration was determined by UV-Visible spectrometry (UV-Vis) at 278 nm (standard curve with mAb A was previously determined at 278 nm to determine extinction coefficient) on the Little Lunatic (Unchained Labs) and the mAb A mass released over time was tracked. Sinks were replaced with fresh PBS when applicable if mAb concentration in all sink samples were above the detection limit.

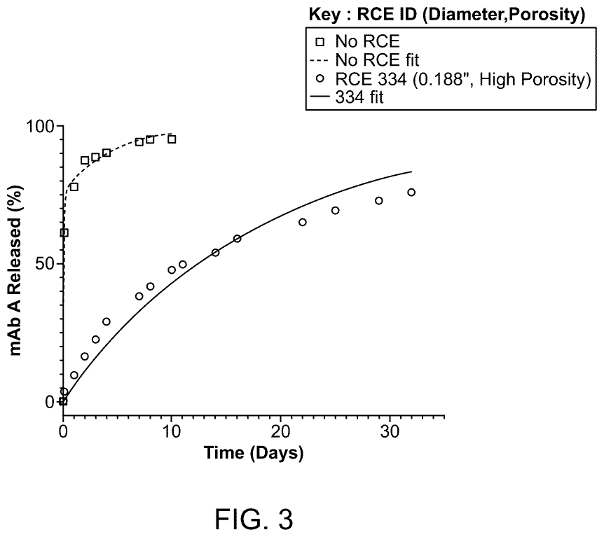

FIG. 3 shows mAb release from devices having a reservoir volume of 4.5 mL and a high porosity RCE compared to no RCE. Each point is the mean of replicate device samples with or without the RCE. Devices with no RCE are shown in open squares with the dotted line being the curve fit to the data (n=5). Devices with a high porosity RCE are shown in open circles with the solid line being the curve fitted to the data (n=4, RCE 334). Exponential models of the mAb release from the devices were fit to the data. The presence of a high porosity RCE having a diameter of 0.188″ affected the mAb release profile compared to no RCE in the device and sustained mAb release over time.

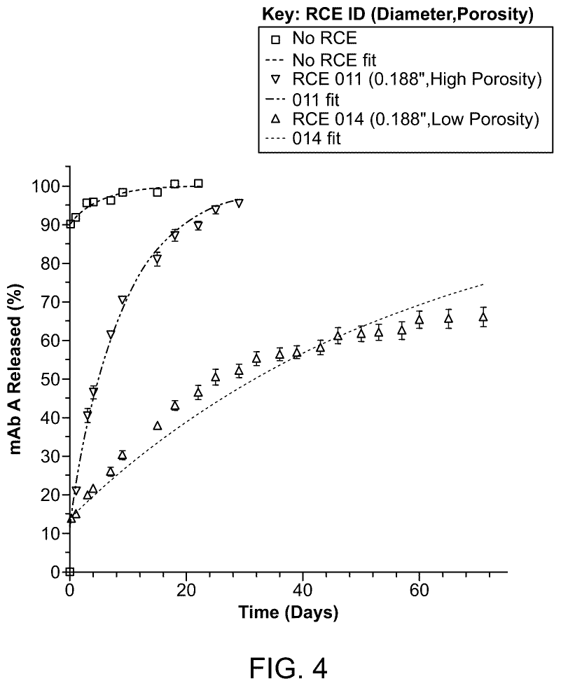

FIG. 4 shows mAb release from devices having a 1 mL reservoir and fit with different RCE types to determine how the RCE properties affect the release of mAb A. Devices were fit with no RCE (open squares), high porosity RCE (open upside down triangle), or low porosity RCE (open triangle). Each of the RCEs had the same diameter and thickness, but different micron grades (i.e., porosity). The decreased pore size of the low porosity RCE slows the release rate of the mAb A. Pore size of the RCE and release rate of the mAb A are proportional.

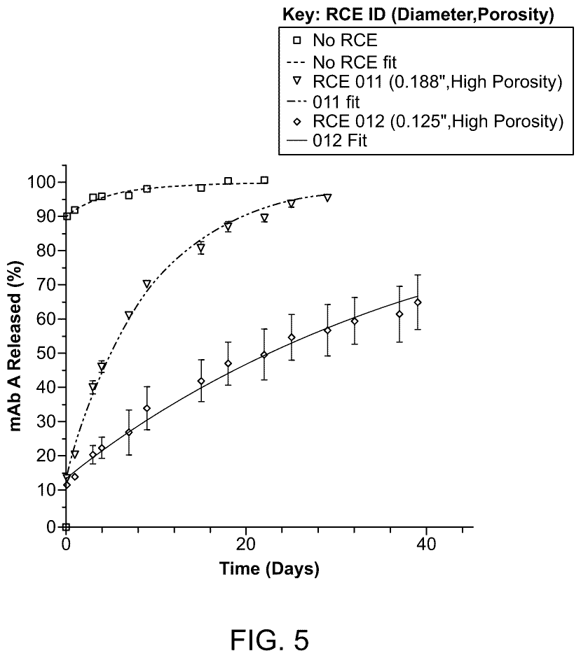

FIG. 5 shows mAb A release from devices having a 1 mL reservoir and fit with no RCE (open squares) or RCEs having the same high porosity, but with different diameters. The larger diameter RCE (open, upside-down triangle) had 0.188″ diameter and the smaller diameter RCE (open diamonds) had 0.125″ diameter. This reduction in diameter of the RCE slowed the release rate of mAb A. There is a proportional relationship between surface area of the RCE and release rate of the mAb A. Thickness was not experimentally tested due to limited RCE selection, however from the laws of diffusion, an inverse relationship between thickness and release rate can be assumed.

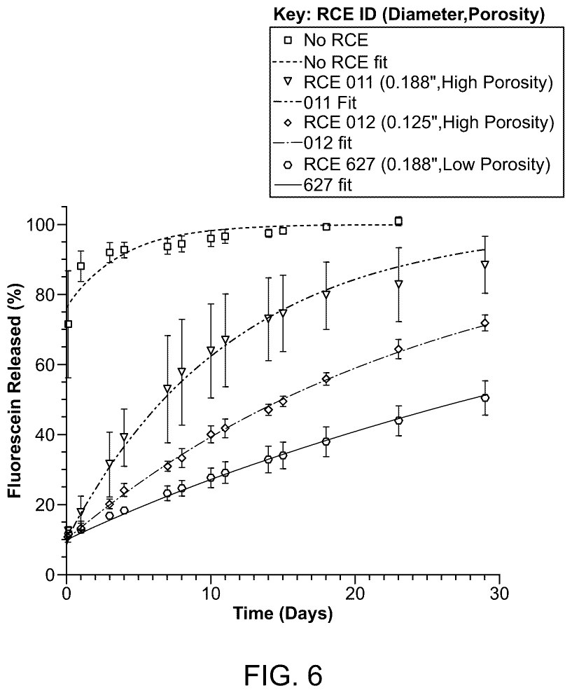

The tests were repeated with a small molecule model of Fluorescein (332 Da). The same trends exist with small molecule models as were present in the large molecule models with mAb A (˜150 kDa). The Fluorescein solution was a 100 mg/mL concentration solution diluted with PBS+0.02% sodium azide (NaN₃) to achieve an initial concentration of 25 mg/mL. The PBS sink volume was adjusted to 200 mL, since Fluorescein has a lower detection capability. A standard curve of absorption at 492 nm was previously determined and this experimental extinction coefficient was used. FIG. 6 shows the Fluorescein release profiles of devices having a 1 mL reservoir and fit with no RCE (open squares) or RCEs having high porosity (open, upside down triangles) or low porosity (open hexagons). One of the high porosity RCEs also had a reduced diameter of 0.125″ (open diamonds) compared to the larger 0.188″ diameter. As with the larger molecule models, the small molecule model shows the decreased porosity slows the release of the Fluorescein and the reduction in surface area slows the release rate of Fluorescein.

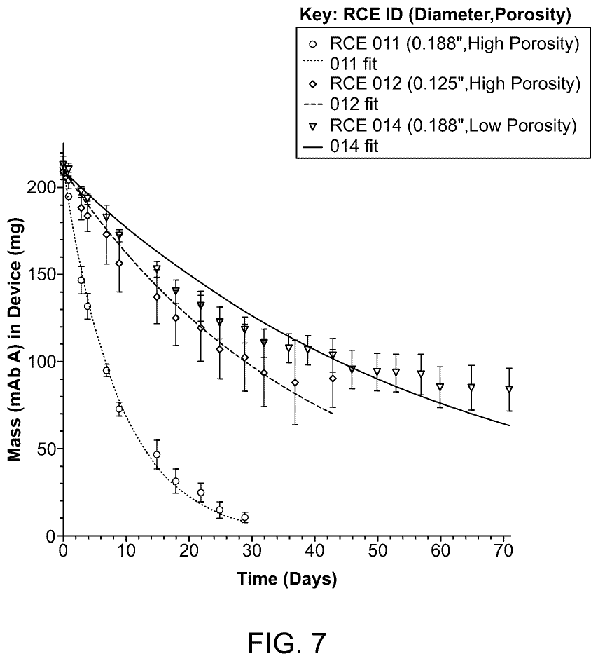

FIG. 7 shows the experimental data points along with each fitted curve (following Eq. 3) coinciding with different RCEs for the small reservoir device (1 mL) filled with large molecular weight mAb A. The fit seems to match well with the data, however, as time progresses the assumption that the concentration in the device is much greater than the concentration in the catheter breaks down slightly. Additionally, in ICV delivery, the sink (e.g., CSF) is replenished by CSF production in the ventricles more quickly than the sink changes that were made experimentally. This will cause a greater concentration gradient and can result in quicker molecular release rates. Using the rate constant k=(D*RRI)/Vdev, this fit parameter can be used to exhibit significance in the molecular release rates. The result showed that RCE 014 (open, upside down triangles) having a low porosity and larger diameter of 0.188″ and RCE 012 (open diamonds) having high porosity and smaller diameter of 0.125″ had significantly different mAb A release rates than RCE 011 (open circles) having high porosity and larger diameter of 0.188″. For this fitting data, time equals zero was started at 3 hours into release because the therapeutic agent was filled into the diffusion area under the RCE to avoid air bubbles. It is assumed that after 3 hours the majority of molecules, which started under the RCE, diffused out of the catheter and into the sink. This assumption is confirmed by the quick release of therapeutic agent in devices without the RCE.

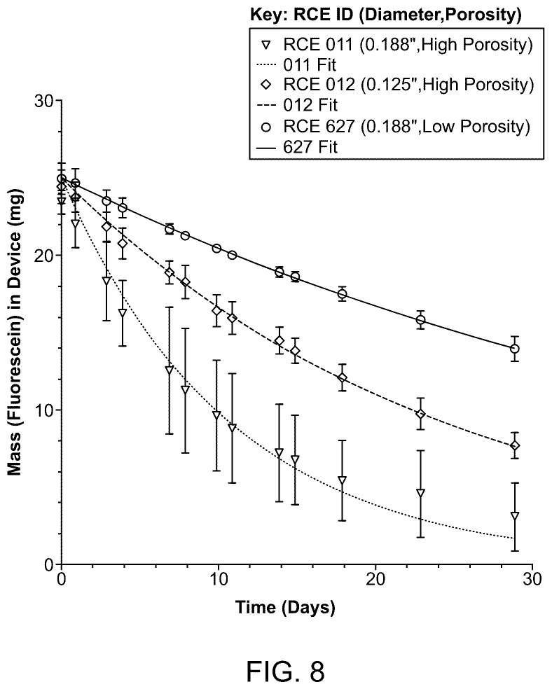

FIG. 8 shows the experimental data points along each fitted curve (following Eq. 3) coinciding with the different RCEs for the small reservoir device (1 mL) filled with small molecular weight Fluorescein. The fit shows good agreement with the experimental data. All three RCEs showed significantly different release rate constants from one another. The result showed that RCE 011 (open, upside down triangles) having a high porosity and larger diameter of 0.188″ and RCE 012 (open diamonds) having high porosity and smaller diameter of 0.125″ had significantly different Fluorescein release rates than RCE 627 (open circles) having low porosity and larger diameter of 0.188″. Since fluorescein could be detected at lower concentrations, the sink size could be greater and possibly better fits the assumptions that diffusion through the RCE is the rate limiting step and that concentration in the device remained much greater than in the catheter and sink.

The data show release of a small molecule model therapeutic agent, Fluorescein, and a large molecule model therapeutic agent, mAb A, in a controlled manner to achieve sustained delivery over time by incorporating a porous metal frit (RCE) into an implantable and refillable ICV reservoir device. RCE parameters (surface area, length, and porosity) can be modified to achieve the desired release rate of therapeutic agents for different clinical applications. The two molecules tested Fluorescein and mAb A bracket other potential classes of therapeutic agents such as peptides, proteins, ASOs (antisense oligonucleotides) and Fab agents (antigen-binding fragments) in terms of molecular size.

### Methods of Use

It should be appreciated that the treatment devices described herein can be used in a variety of locations and implanted in a variety of ways. The implantation method and use of the treatment devices described herein can vary depending on the type of treatment device being implanted and the intended location and drug for treatment. As will be described in more detail below, the treatment devices described herein can be primed, implanted, filled, refilled, and/or explanted using one or more devices.

In one implementation of treatment device implantation, a skin flap can be formed in the scalp to expose a region of the skull that is located directly over a target delivery location. A burr hole can be formed in the skull to expose a region of the dura. The burr hole is typically drilled to a depth and diameter that mirrors the shape of the lower end of the container. The diameter of the base of the burr hole is sized to accommodate the lower surface 115 of the base 114 to allow for recessed positioning of the device. The ideal trajectory of catheter implantation can be performed using neuronavigation software and imaging convention is the implantation of devices such as the Ommaya reservoir (see www.cureus.com/articles/29046-ommaya-reservoir-insertion-a-technical-note).

The catheter 110, which may have been cut to size at the time of implantation, can be affixed to the barb 128 on a lower end of the device 100. The catheter can be cut just short of the distance of the planned trajectory to account for the depth of the barbed connector that inserts into the burr hole. The container 105 can be filled with the drug to be delivered at the time of implantation or provided pre-filled with the drug. The catheter 110 can be primed prior to coupling to the barb 128 so as to be filled with BSA and any air bubbles released. The distal tip 112 of the catheter 110 can be inserted through the burr hole and advanced to the target location, such as within the ventricle below the burr hole. The upper region 123 of the barbed connector 127 can be positioned within the burr hole and the lower surface 115 of the base 114 positioned against the skull surface. The flap of skin can be positioned back over the upper surface 117 of the cap 116 and sutured. Thus, the container 105 remains outside the skull and under the scalp so as to be easily refilled by needle penetration. The device implantation procedure can be similar to the procedure for implanting an Ommaya reservoir (see www.cureus.com/articles/29046-ommaya-reservoir-insertion-a-technical-note).

Generally, the implementations of the treatment devices described herein contain drug solutions, drug suspensions and/or drug matrices. The treatment devices described herein can also contain therapeutic agents formulated as one or more solid drug core or pellets formulated to deliver the one or more therapeutic agents at therapeutically effective amounts for an extended period of time. The period of time over which the treatment device delivers therapeutically effective amounts can vary. In some implementations, the treatment device is implanted to provide a therapy over the effective life of the device such that refill of the device is not necessary.

The treatment devices described herein need not be removed and can remain in place indefinitely so long as therapeutically effective and beyond. However, the treatment device 100 can be explanted (i.e. removed from the target location).

### Indications

The treatment devices described herein can be used to treat and/or prevent a variety of neurodegenerative diseases of the brain including Alzheimer's disease, stroke, Huntington's disease, amyotrophic lateral sclerosis (ALS), Angelman syndrome, Parkinson's disease, motor neuron disease, and other diseases of the brain including brain cancer, Batten disease such as late infantile neuronal ceroid lipofuscinosis type 2 (CLN2) also known as tripeptidyl peptidase 1 (TPP1) deficiency, CNS trauma, and other diseases.

### Therapeutics

Examples of therapeutic agents that may be delivered by the treatment devices described herein include but are not limited to antisense oligonucleotides, CKPs, various immunotherapies such as monoclonal antibodies, as well as thrombolytics, protease inhibitors and others useful for treating various neurodegenerative disorders. Therapeutic agents listed in Bhavna Kumar, et al., “Recent Patent Advances for Neurodegenerative Disorders and its Treatment”, Recent Patents on Drug Delivery & Formulation (2017) 11 (3): 158-172 are considered as well. Other therapeutic agents known to those skilled in the art which are capable of controlled, sustained release into the patient in the manner described herein are also suitable for use in accordance with embodiments of the devices described herein.

### Materials

Generally, the components of the devices described herein are fabricated of materials that are biocompatible and preferably insoluble in the body fluids and tissues that the device comes into contact with. The materials generally do not cause irritation to the portion of the tissue that it contacts. Materials may include, by way of example, various polymers including, for example, silicone elastomers and rubbers, polyolefins, polyurethanes, acrylates, polycarbonates, polyamides, polyimides, polyesters, and polysulfones.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various placed throughout this specification are not necessarily referring to the same embodiment or implementation. Furthermore, the particular features, structures, configurations, or characteristics may be combined in any suitable manner in one or more implementations.

The devices and systems described herein can incorporate any of a variety of features. Elements or features of one implementation of a device and system described herein can be incorporated alternatively or in combination with elements or features of another implementation of a device and system described herein. For the sake of brevity, explicit descriptions of each of those combinations may be omitted although the various combinations are to be considered herein. Additionally, the devices and systems described herein can be positioned in the patient and need not be implanted specifically as shown in the figures or as described herein. The various devices can be implanted, positioned and adjusted etc. according to a variety of different methods and using a variety of different devices and systems. The various devices can be adjusted before, during as well as any time after implantation. Provided are some representative descriptions of how the various devices may be implanted and positioned, however, for the sake of brevity explicit descriptions of each method with respect to each implant or system may be omitted.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “upper,” “lower,” “top”, “bottom,” “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

What is claimed is:

1. A device for delivery of a drug into cerebrospinal fluid of a brain, the device comprising:
a hollow container having a reservoir volume and an outlet, the container comprising an upper surface and a lower surface;
a porous structure comprising a sintered rigid material that passively regulates diffusion of a drug from the container at a controlled release rate; and
a catheter coupled to a lower end of the container, the catheter having a lumen, a proximal end region, and a distal end region, the lumen configured to communicate with the reservoir volume of the container through the porous structure.

2. The device of claim 1, wherein the container is sized for implantation between a region of the skull and overlying skin.

3. The device of claim 2, wherein, when implanted, the lower surface of the container is positioned against the skull and the overlying skin covers the upper surface of the container.

4. The device of claim 3, wherein the lower surface of the container is flat or has a curved profile that conforms substantially to a shape of the skull at the region.

5. The device of claim 1, wherein the container is about 10 mm to about 45 mm in diameter.

6. The device of claim 1, wherein a maximum height of the container between the lower surface and the upper surface is about 3 mm to about 15 mm.

7. The device of claim 1, wherein the container is oval, elliptical or circular in perimetrical shape.

8. The device of claim 1, wherein the reservoir volume is about 0.5 mL to about 5.0 mL.

9. The device of claim 1, wherein the container comprises a lower base having the lower surface and an upper cap having the upper surface, the base and cap together defining the reservoir volume.

10. The device of claim 9, wherein at least a portion of the cap is formed of a material configured to be penetrated by a needle for injection of the drug.

11. The device of claim 9, wherein the base defines the outlet from the container.

12. The device of claim 9, wherein the base has an internal surface that includes a seat configured to seal with the porous structure.

13. The device of claim 12, further comprising a diffusion chamber located distal to the porous structure within the seat, the diffusion chamber comprising an upper region and a lower region.

14. The device of claim 13, wherein the diffusion chamber is located within a projection that projects from the lower surface of the base.

15. The device of claim 13, wherein the porous structure has an upper surface facing the reservoir volume of the container and a lower surface facing the diffusion chamber.

16. The device of claim 13, wherein the upper region of the diffusion chamber has an inner diameter that is less than an inner diameter of the container.

17. The device of claim 16, wherein an outlet channel is located below the lower region of the diffusion chamber, the outlet channel having an inner diameter that is less than an inner diameter of the lower region.

18. The device of claim 17, wherein the lower region of the diffusion chamber tapers from the inner diameter of the upper region to the inner diameter of the outlet channel.

19. The device of claim 17, wherein the outlet channel is positioned within a barbed connector of the container, the barbed connector configured to couple the catheter to the container.

20. The device of claim 19, wherein a barb of the barbed connector mitigates unintentional dislodgement between the catheter and the container.

21. The device of claim 20, wherein the barb of the barbed connector has an outer diameter that is larger than an inner diameter of the lumen of the catheter.

22. The device of claim 19, wherein the barbed connector and base are formed of a material that is more rigid than the proximal end region of the catheter.

23. The device of claim 21, wherein when the device is implanted in a patient, the barbed connector positioned within the lumen of the catheter is positioned internal to the skull and the container is positioned external to the skull.

24. The device of claim 1, wherein the catheter is straight between proximal and distal ends upon implantation so that the container is positioned above the distal end of the catheter when the distal end is positioned within a target site.

25. The device of claim 1, wherein the catheter has a length sufficient to extend to a target location in the brain.

26. The device of claim 25, wherein the length is about 3 cm to about 15 cm.

27. The device of claim 25, wherein the length of the catheter is no greater than 11 cm.

28. The device of claim 25, wherein the target location is a dural sinus or a ventricle of the brain.

29. The device of claim 25, wherein the length of the catheter is longer than a distance between the skull and the target location.

30. The device of claim 29, wherein the proximal end region of the catheter is configured to be cut to size prior to implantation of the container.

31. The device of claim 30, wherein the length of the catheter is between 30 cm and 50 cm and the distance is about 3 cm to about 15 cm.

32. The device of claim 30, wherein an outer surface of the catheter at the proximal end region comprises one or more marks indicating a length of the catheter to a distal-most end of the catheter.

33. The device of claim 1, wherein the distal end region of the catheter has at least one opening from the lumen.

34. The device of claim 33, wherein the at least one opening of the distal end region of the catheter includes a plurality of outlets positioned through a wall of the catheter.

35. The device of claim 34, wherein the plurality of outlets are about 0.02 mm to about 2 mm in diameter.

36. The device of claim 34, wherein each outlet of the plurality of outlets are spaced angularly at about 90 degrees from each other so as to occur circumferentially around the distal end region of the catheter.

37. The device of claim 34, wherein each outlet of the plurality of outlets are spaced apart by about 45, 90, 120, or 180 degrees.

38. The device of claim 1, wherein the catheter has a cross-sectional outer diameter that is no more than 5 mm.

39. The device of claim 38, wherein the cross-sectional outer diameter of the catheter is about ⅛″ (3.175 mm) and an inner diameter of the catheter is about 1/16″ (1.5875 mm).

40. The device of claim 1, wherein the porous structure is made of titanium or stainless steel.

41. The device of claim 1, wherein the porous structure has a porosity that is about 1% to about 70%.

42. The device of claim 1, wherein the drug is released from the container for at least 1 month up to about 36 months.

43. The device of claim 1, wherein the drug has a molecular weight of at least about 100 Daltons up to about 200,000 Daltons.

44. The device of claim 1, wherein the drug is a peptide, a protein, an antisense oligonucleotide, or antigen-binding fragment.

45. The device of claim 1, wherein the drug is useful for treating a neurodegenerative disease of the brain.

46. The device of claim 1, wherein the drug is useful for treating Alzheimer's disease, stroke, Huntington's disease, amyotrophic lateral sclerosis (ALS), Angelman syndrome, Parkinson's disease, motor neuron disease, brain cancer, neuronal ceroid lipofuscinosis, tripeptidyl peptidase 1 (TPP1) deficiency, or central nervous system trauma.

47. A method of controlling the delivery of a drug to an internal portion of a body comprising administering the drug to the internal portion through a device according to claim 1.

48. A method of treating a disease of a brain in a subject in need thereof, the method comprising:
administering to the subject an effective amount of a drug into the cerebrospinal fluid (CSF) with an implantable device, the implantable device comprising:
a hollow container having a reservoir volume and an outlet;
a porous structure comprising a sintered rigid material that passively regulates diffusion of the drug from the container at a controlled rate of release; and
a catheter coupled to a lower end of the container having a lumen configured to communicate with the reservoir volume of the container through the porous structure.

49. The method of claim 48, wherein the disease of the brain is a neurodegenerative disease.

50. The method of claim 49, wherein the neurodegenerative disease is Alzheimer's disease, stroke, Huntington's disease, amyotrophic lateral sclerosis (ALS), Angelman syndrome, Parkinson's disease, or motor neuron disease.

51. The method of claim 48, wherein the disease is brain cancer.

52. The method of claim 48, wherein the drug is an antisense oligonucleotide, a cysteine knot peptide, or a Fab antibody fragment.

53. The method of claim 48, further comprising positioning a distal end region of the catheter within a ventricle of the brain or a dural sinus.

54. The method of claim 48, wherein the sintered rigid material comprises at least one of a metal, a ceramic, and a glass.

55. The method of claim 48, wherein the porous structure and the container are tuned to release a predetermined rate profile of the drug from the reservoir volume into the CSF to treat the brain for an extended period of time.

56. The method of claim 55, wherein the porous structure comprises a porosity, a thickness, a channel parameter and a surface area configured to release therapeutic amounts of the drug for the extended period of time.

57. The method of claim 56, wherein the channel parameter comprises a fit parameter corresponding to an effective length of a plurality of irregularly shaped channels extending from a first side of the porous structure to a second side of the porous structure.

58. The method of claim 57, wherein the rate of release of the drug through the porous structure corresponds to a ratio of the porosity to the channel parameter and wherein the ratio of the porosity to the channel parameter is between 0.03 and 0.2 such that the porous structure is capable of releasing the therapeutic agent for the extended period.
